(19) United States
(12) Patent Application Publication
(10) Pub. No.: US 2009/0291509 A1
(43) Pub. Date: Nov. 26, 2009

(54) Sensing method

(75) Inventor: Shunichi Wakamatsu, Sayama-shi (JP)

Correspondence Address: JORDAN AND HAMBURG LLP, 122 EAST 42ND STREET, SUITE 4000, NEW YORK, NY, 10168, US

(73) Assignee: NIHON DEMPA KOGYO CO., LTD., Shibuya-ku (JP)

(21) Appl. No.: 12/454,434

(22) Filed: May 18, 2009

(30) Foreign Application Priority Data
May 19, 2008 (JP) 2008-131262

(51) Int. Cl. G01N 33/531

(52) U.S. Cl. 436/543

(57) ABSTRACT

To enable successive measurement of samples by using one piezoelectric sensor. An adsorption layer provided in a piezoelectric sensor is an adsorption layer in which protein and an immunoglobulin are stacked in this order from the bottom. The immunoglobulin has a property of separating from protein when coming into contact with an acid liquid, and therefore, when the immunoglobulin as the adsorption layer is brought into contact with the acid liquid after capturing a substance to be sensed, the immunoglobulin is separated from the protein. Then, the adsorption layer is again formed by the supply of a new immunoglobulin, which enables the successive measurement of samples.

### BACKGROUND OF THE INVENTION

1. Field of the Invention

The present invention relates to a sensing method for recognizing and quantifying a substance to be sensed contained in a sample solution, based on a frequency of a piezoelectric resonator such as a quartz resonator.

2. Description of the Related Art

As a piezoelectric sensor sensing and measuring a trace substance contained in a sample solution, there has been known a quartz sensor using a quartz resonator. In this quartz sensor, an adsorption layer made of a biological substance film or the like recognizing and reacting with a specific substance to be sensed is formed on a front surface of a metal electrode (excitation electrode) provided on a quartz piece. When reacting with the substance to be sensed present in a sample solution to adsorb the substance to be sensed, the adsorption layer changes in mass, which causes a change in natural frequency of the quartz resonator. By utilizing this operation, the concentration of the substance to be sensed is measured.

As the biological substance, used is, for example, a film of an antibody reacting with a specific antigen, and the film of the antibody adsorbs the antigen. A method of fabricating such a quartz sensor is as follows. A buffer solution is supplied into the quartz sensor including therein the quartz resonator, and then a solution containing a predetermined amount of antibody is supplied into the quartz sensor, so that the antibody is adsorbed by the front surface of the metal electrode of the quartz resonator. Next, a solution containing a predetermined amount of a substance for blocking (blocker) made of, for example, protein is injected into the quartz sensor so that the front surface of the metal electrode adsorbs the blocker. A reason why the front surface of the metal electrode is made to adsorb the blocker is to prevent the antigen from being adsorbed by the front surface of the metal electrode, thereby forming an environment where the antigen is adsorbed only by the antibody, and to ensure high accuracy in a correspondence relation between an amount of the antigen captured by the antibody and the frequency.

However, since it is difficult to separate the antigen and the antibody once the antigen and the antibody react with and bind to each other, the quartz sensor using the antigen-antibody reaction is usable only once and then is discarded. This requires a troublesome work of replacing the quartz sensor every time the sample is changed and also is a waste of resources.

Patent document 1 describes a measuring device including eight quartz sensors which are attachably/detachably provided in a measuring device main body measuring a frequency change of a quartz resonator, thereby facilitating a work of measuring the concentration of a substance to be sensed and reducing the time for the measuring work. However, this art is not capable of solving the aforesaid problem since the quartz sensors are discarded after use.

[Patent Document 1] Japanese Patent Application Laid-open No. 2006-194868 (paragraph 0012 and FIG. 1)

### SUMMARY OF THE INVENTION

The present invention was made under such circumstances and has an object to provide a sensing method capable of measuring a sample a plurality of times with one piezoelectric sensor.

The sensing method may be structured as follows.

A sensing method of the present invention is a method in which a piezoelectric resonator having an excitation electrode formed on a piezoelectric piece is oscillated by an oscillator circuit while being in contact with a liquid and an antigen in a sample solution is sensed based on a change in natural frequency of the piezoelectric resonator,

the piezoelectric resonator being a piezoelectric resonator in which a base layer made of protein adsorbing an immunoglobulin and desorbing the immunoglobulin under an atmosphere of an acid liquid is formed on the excitation electrode, the method including:

a step of supplying a liquid containing the immunoglobulin to the piezoelectric resonator to make the base layer adsorb the immunoglobulin;

a first measuring step of oscillating the piezoelectric resonator which has adsorbed the immunoglobulin and measuring a frequency corresponding to an oscillation frequency of the oscillator circuit;

a step, which follows the first measuring step, of supplying the piezoelectric resonator with the sample solution containing the antigen which is a substance to be sensed captured by the immunoglobulin;

a second measuring step, which follows the step of supplying the sample solution, of oscillating the piezoelectric resonator which has adsorbed the antigen and measuring a frequency corresponding to an oscillation frequency of the oscillator circuit;

a step of finding a difference between the frequencies obtained in the first measuring step and the second measuring step respectively; and

a step, which follows the step of finding the difference, of supplying the acid liquid to the piezoelectric resonator to make the base layer desorb the immunoglobulin.

A sensing method according to another aspect of the present invention is a method in which a piezoelectric resonator having an excitation electrode formed on a piezoelectric piece is oscillated by an oscillator circuit while being in contact with a liquid and an immunoglobulin in a sample solution is sensed based on a change in natural frequency of the piezoelectric resonator,

the piezoelectric resonator being a piezoelectric resonator in which a base layer made of protein adsorbing the immunoglobulin and desorbing the immunoglobulin under an atmosphere of an acid liquid is formed on the excitation electrode, the method including:

a step of supplying the piezoelectric resonator with the sample solution containing the immunoglobulin which is a substance to be sensed to make the base layer adsorb the immunoglobulin;

a first measuring step of oscillating the piezoelectric resonator which has adsorbed the immunoglobulin and measuring a frequency corresponding to an oscillation frequency of the oscillator circuit;

a step, which follows the first measuring step, of supplying the piezoelectric resonator with a liquid containing an antigen captured by the immunoglobulin;

a second measuring step of oscillating the piezoelectric resonator which has adsorbed the antigen and measuring a frequency corresponding to an oscillation frequency of the oscillator circuit;

a step of finding a difference between the frequencies obtained in the first measuring step and the second measuring step respectively; and

a step, which follows the step of finding the difference, of supplying the acid liquid to the piezoelectric resonator to make the base layer desorb the immunoglobulin.

The sensing method may further include a step of supplying a liquid containing the immunoglobulin to the piezoelectric resonator which has adsorbed the antigen to make the antigen adsorb the immunoglobulin, so as to sandwich the antigen between the immunoglobulin and the immunoglobulin which has already adsorbed the antigen, wherein

the second measuring step may be a step of measuring the frequency while the antigen is sandwiched by the immunoglobulins.

A self-assembled monolayer may be formed between the excitation electrode and the base layer. Further, a blocking substance for preventing the adsorption of the antigen may be adsorbed by a portion, on the excitation electrode, which has not adsorbed the immunoglobulin.

In the present invention, protein which has a property of reacting specifically with an immunoglobulin and desorbing the immunoglobulin when it comes into contact with the acid liquid is attached as the base film on the electrode of the piezoelectric resonator, and the immunoglobulin is made to capture the antigen by an antigen-antibody reaction or the antigen is further made to capture the immunoglobulin. Then, one of the immunoglobulin and the antigen is sensed as a substance to be sensed based on a change in the frequency. Then, the base film is made to desorb the immunoglobulin by the supply of the acid liquid after the measurement of the frequency. Therefore, it is possible to reproduce a new adsorption layer made of the immunoglobulin by repeating a series of the processes, which makes it possible to conduct the measurement a plurality of times with one piezoelectric sensor. As a result, in a work of preparing a calibration curve or in a work of measuring a substance to be sensed by using a calibration curve, it is possible to reduce running cost and to save the trouble of changing piezoelectric sensors every time a sample is measured.

### BRIEF DESCRIPTION OF THE DRAWINGS

FIG. 1 is an exterior perspective view showing a sensor unit of a sensing instrument according to an embodiment of the present invention;

FIG. 2 is an exploded perspective view showing upper surface sides of components of the sensor unit;

FIG. 3 is a view showing a vertical cross section of the sensor unit;

FIG. 4 is a vertical sectional view of a quartz resonator included in the quartz sensor;

FIG. 5 is a view schematically showing an adsorption layer formed on the quartz resonator;

FIG. 6 is a diagram schematically showing the whole structure of the sensing instrument;

FIG. 7(a) to FIG. 7(c) are schematic views showing how the adsorption layer changes by liquid supply;

FIG. 8 is a chart showing one example of the correlation between frequency and concentration of immunoglobulin; and

FIG. 9 is a schematic explanatory view showing how the immunoglobulin is adsorbed by an antigen after the antigen is adsorbed by the adsorption layer.

### DETAILED DESCRIPTION OF THE PREFERRED EMBODIMENTS

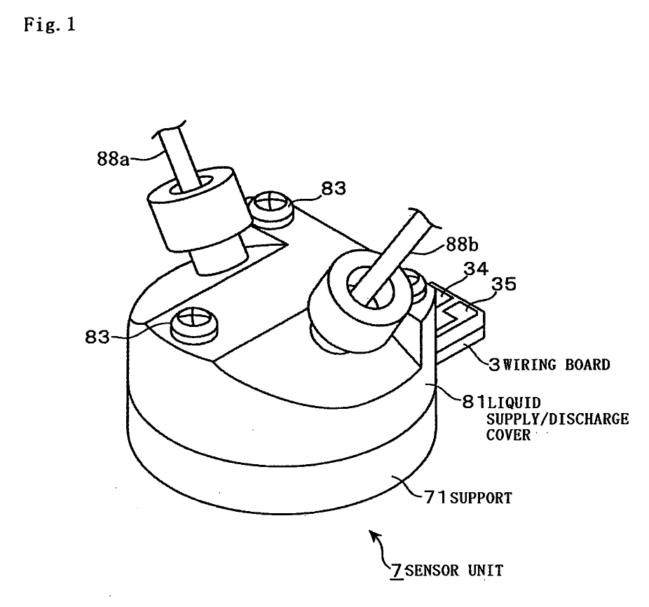

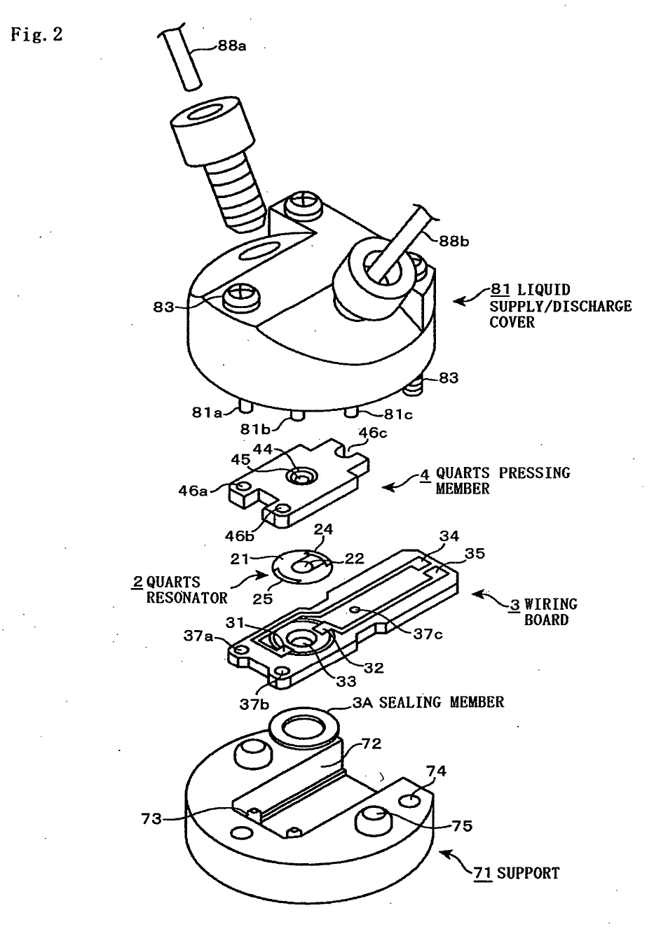

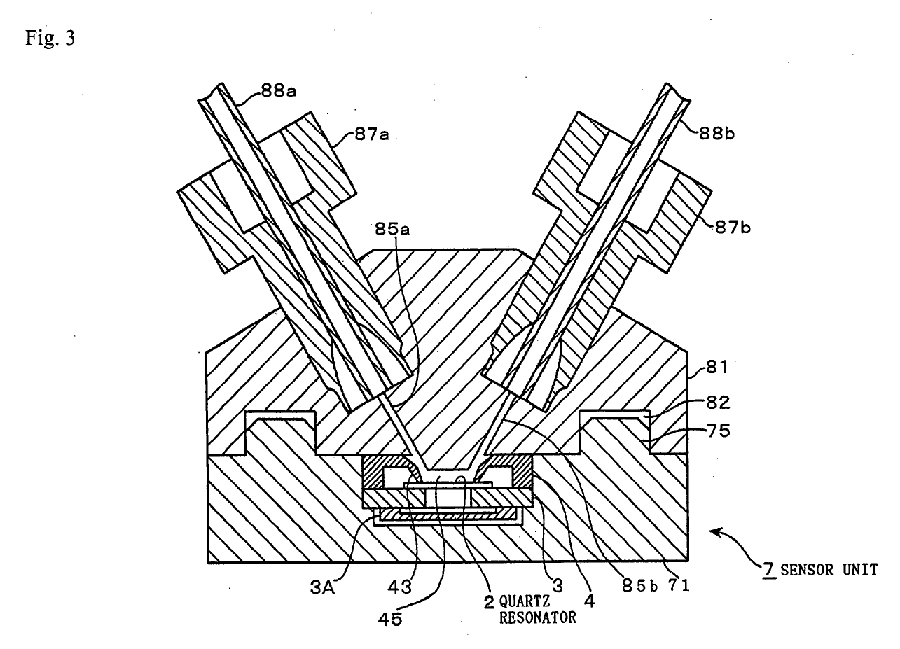

An embodiment of a sensing instrument according to the present invention will be described by using the drawings. FIG. 1 is an exterior perspective view showing a sensor unit of the sensing instrument, FIG. 2 is an exploded perspective view showing upper surface sides of components of the sensor unit, and FIG. 3 is a view showing a vertical cross section of the sensor unit. As shown in FIG. 2, the sensor unit includes a support 71, a sealing member 3A, a wiring board 3, a quartz resonator 2, a quartz pressing member 4, and a liquid supply/discharge cover 81, and these components are stacked from the bottom in this order.

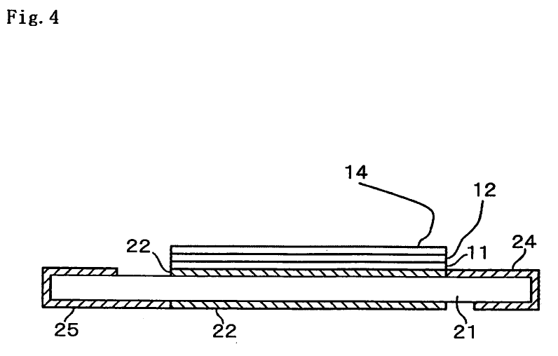

As shown in FIG. 2 and FIG. 4, the quartz resonator 2 as a piezoelectric resonator includes a circular quartz piece 21 as a piezoelectric piece, excitation electrodes 22, and leading electrodes 24, 25. On a front surface side of the quartz piece 21, the excitation electrode 22 in foil form having a circular shape smaller in diameter than the quartz piece 21 is formed, and one end side of the leading electrode 24 in foil form is connected to the excitation electrode 22. The leading electrode 24 is bent along an end surface of the quartz piece 21 to extend along a rear surface side of the quartz piece 21. On the rear surface side of the quartz piece 21, the excitation electrode 22 and the leading electrode 25 are formed to be connected to each other in the same layout as that on the front surface side. An equivalent thickness of the excitation electrodes 22 and the leading electrodes 24, 25 is, for example, 0.2 μm, and for example, gold, silver, or the like is used as a material of the electrodes.

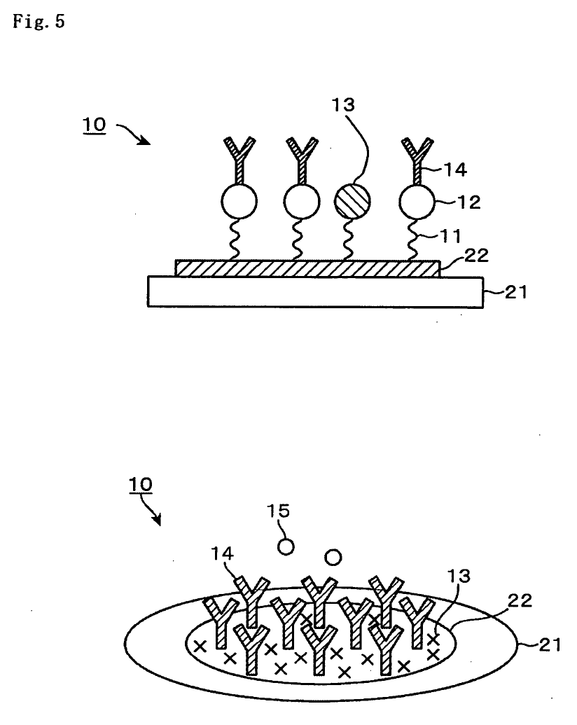

On the excitation electrode 22 on the front surface side (side in contact with a sample solution) provided on the quartz piece 21, an adsorption layer 10 adsorbing a substance to be sensed is formed. A method of forming the adsorption layer 10 will be described by using FIG. 5. First, a self-assembled monolayer 11 is formed on the excitation electrode 22 on the side in contact with the sample solution. The self-assembled monolayer 11 is formed in such manner that the quartz resonator 2 is brought into contact with a solution of organic molecules and the organic molecules are chemically adsorbed by the excitation electrode 22. As the self-assembled monolayer 11, usable is, for example, 3.3-dithidipropionic acid (N-hydroxysuccinimide). The function of the self-assembled monolayer 11 is to make protein and the like bind to terminals of the molecules to realize higher binding efficiency than when the protein and the like are made to directly bind to the excitation electrode 22, thereby enabling stable film formation. Subsequently, the self-assembled monolayer 11 is activated by using NHS/EDC (N-hydroxysuccinimide/ethane dichloride), and thereafter, for example, protein A as the protein is supplied to a front surface of the self-assembled monolayer 11, thereby forming a base layer 12 made of the protein.

Next, for example, an ethanolamine solution is supplied to the self-assembled monolayer 11, so that a blocker 13 as ethanolamine binds to the self-assembled monolayer 11. Further, an immunoglobulin 14 such as IgG which is an antibody is adsorbed by the base layer 12 made of the protein, whereby the adsorption layer 10 made of the immunoglobulin is formed. The immunoglobulin 14 is capable of capturing a substance to be sensed (antigen) 15 in a sample solution. Further, the immunoglobulin 14 has a property of reacting specifically with, for example, the protein A and separating from the protein when brought into contact with an acid liquid.

Next, the sensor unit 7 will be described with reference to FIG. 2. The wiring board 3 is formed by, for example, a printed circuit board, and on a front surface thereof, an electrode 31 and an electrode 32 are provided at an interval. As will be described later, between the electrodes 31, 32, a through hole 33 is formed to serve as a recessed portion forming an airtight space faced by the excitation electrode 22 on the rear surface side of the quartz resonator 2, and the through hole 33 has a diameter large enough to house the excitation electrode 22. On a rear end side of the wiring board 3, connection terminal portions 34, 35 are provided to be electrically connected to the electrodes 31, 32 via conductive paths respectively. The sealing member 3A is a circular member with a recessed portion formed at a center thereof, and has a function of closing a lower surface of the through hole 33 to form the airtight space which is a rear surface-side atmosphere of the quartz resonator 2.

The quartz pressing member 4 is formed in a shape corresponding to the wiring board 3 by using an elastic material, for example, silicon rubber. As shown in FIG. 3, on a lower surface of the quartz pressing member 4, an annular projection 43 is provided to press a peripheral portion of the excitation electrodes 22 of the quartz resonator 2 toward the support 71 and to demarcate and form a liquid receiving space above the excitation electrode 22. The function of the annular projection 43 is to fix the positions of the quartz pressing member 4, the quartz resonator 2, and the wiring board 3 by pressing the quartz resonator 2 to an outer area of the through hole 33 formed in the wiring board 3. Further, an opening 44 is formed in an upper surface of the quartz pressing member 4 to communicate with the space surrounded by the annular projection 43. Further, an area surrounded by a peripheral side surface of the opening 44 and an upper surface of the quartz resonator 2 forms an area which is not only an area where the sample solution is brought into contact with the excitation electrode 22 on the front surface of the quartz resonator 2 but also a liquid storage space 45 storing the sample solution.

As shown in FIG. 3, recessed portions 82 formed in a lower surface of the liquid supply/discharge cover 81 are mated with projections 75 provided on the support 71 side, so that the liquid supply/discharge cover 81 is positioned relative to the support 71, and the liquid supply/discharge cover 81 is fixed by its screws 83 being screwed to holes 74 in the support 71 side. In this manner, the liquid supply/discharge cover 81 functions to press the quartz pressing member 4 to the wiring board 3 while the wiring board 3 is housed in a recessed portion 72 provided in the support 71, thereby causing the annular projection 43 to press the quartz resonator 2 to the wiring board 3 to fix the position of the quartz resonator 2.

Further, as shown in FIG. 3, in the liquid supply/discharge cover 81, liquid channels 85a, 85b are slantly provided to communicate with the liquid storage space 45. 88a and 88b in FIG. 3 are a liquid supply pipe and a liquid discharge pipe respectively, which communicate with the channels 85a, 85b respectively. 87a denotes a screw member forming a supply port. The screw member 87a has a through hole in whose center the liquid supply pipe 88a is inserted, and when screwed to the liquid supply/discharge cover 81 side, it fixes the liquid supply pipe 88a and prevents liquid leakage. Further, a screw member 87b forming a discharge port, which is similarly structured, fixes the liquid discharge pipe 88b and prevents liquid leakage.

The support 71 has the recessed portion 72 housing and holding the wiring board 3, and in the recessed portion 72, engagement projections 73 extend in a vertical direction to be engaged with engagement holes 37a, 37b of the wiring board 3 and engagement holes 46a, 46b of the quartz pressing member 4, thereby fixing the positions of the wiring board 3 and the quartz pressing member 4.

In the above, the quartz resonator 2, the wiring board 3, and the sealing member 3A correspond to a piezoelectric sensor of the present invention. The rear surface side of the quartz resonator 2 is exposed to the airtight atmosphere, and therefore, the piezoelectric sensor forms a Languban-typed quartz sensor.

Next, the whole structure of the sensing instrument according to the embodiment of the present invention will be described. The sensing instrument includes the sensor unit 7, an oscillator circuit 50, a measuring circuit part 51, a display device 52, a buffer solution supply part 53, an immunoglobulin-containing liquid supply part 54, a sample solution supply part 55, an acid liquid supply part 58, a supply liquid switching part 63, a waste liquid storage part 56, a pump 62, and a control unit 100.

The oscillator circuit 50 is electrically connected to the electrodes 34, formed in the wiring board 3. The measuring circuit part 51 analog/digital-converts (A/D-converts) a frequency signal sent from the oscillator circuit 50 and applies predetermined signal processing to the resultant signal to measure a frequency of the frequency signal. The oscillator circuit 50 and the measuring circuit part 51 are provided in different casings respectively, and these casings are connected by a cable.

The buffer solution supply part 53, the immunoglobulin-containing liquid supply part 54, the sample solution supply part 55, and the acid liquid supply part 58 are connected to the supply liquid switching part 63 via supply channels 57a, 57b, 57c, 57d respectively. The supply liquid switching part 63 is connected to the liquid supply pipe 88a and has a function of switching a supply channel to be connected to the liquid supply pipe 88a, among the supply channels 57a to 57b. Further, the pump 62 is used to discharge a liquid in the sensor unit 7 to the waste liquid storage part 56 via the liquid discharge pipe 88b and a discharge channel 57e. The supply liquid switching part 63 and the pump 62 are controlled by the control part 100, and the control part 100 outputs a control signal so that a one-cycle operation to be described later is performed based on a computer program.

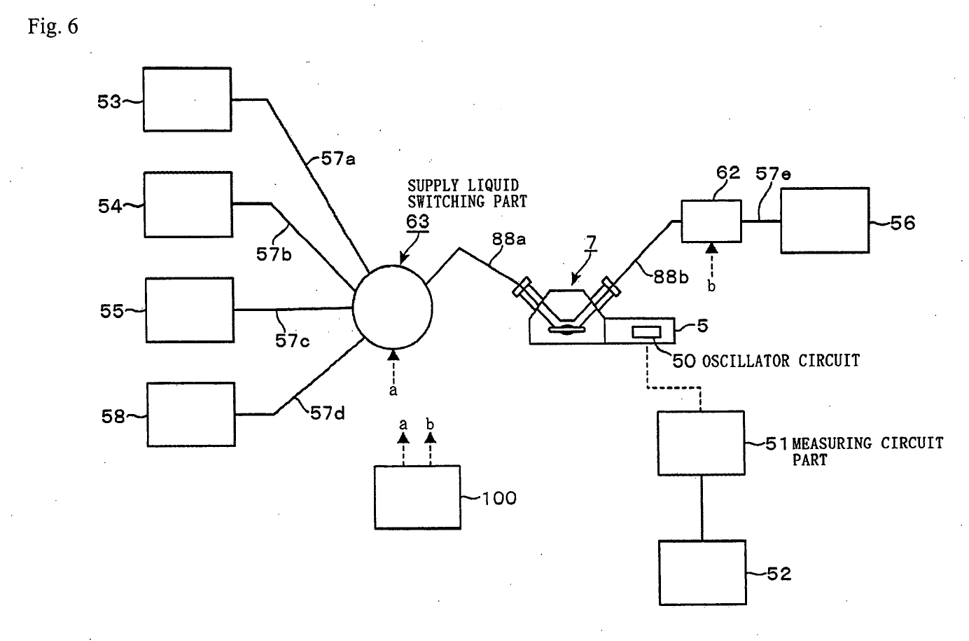

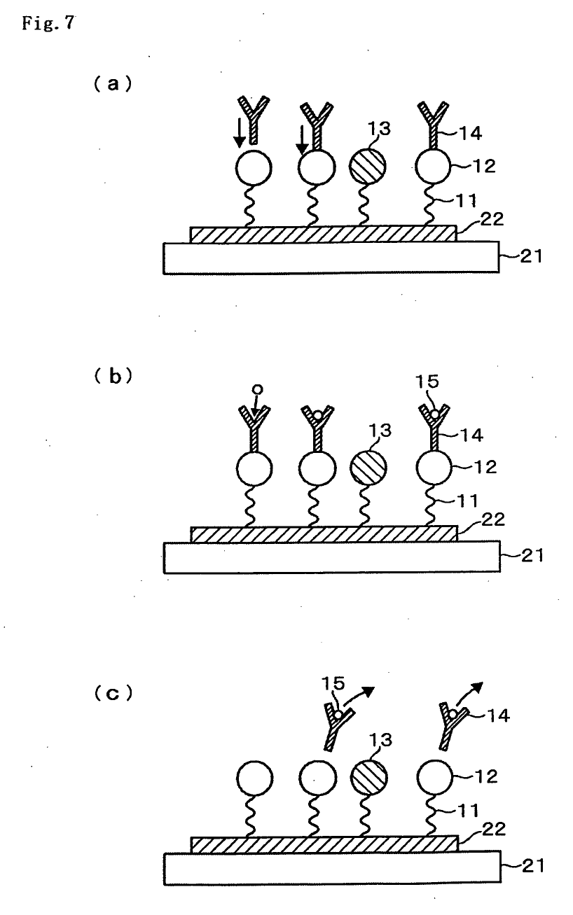

Next, the operation of the sensing instrument as structured above will be described with reference to FIG. 6 and FIGS. 7(a) to 7(c). It is assumed here that the base layer 12 made of protein is formed on the quartz resonator 2 of the piezoelectric sensor. First, the buffer solution supply part 53 supplies a buffer solution to the sensor unit 7. Concretely, the buffer solution is supplied to the liquid storage part 45 inside the sensor unit 7 via the supply channel 57a, the supply liquid switching part 63, and the liquid supply pipe 88a, and further is discharged from the liquid storage part 45 to the waste liquid storage part 56 via the liquid discharge pipe 88b, the pump 62, and the discharge channel 57e. As the buffer solution, phosphor buffer is used, for instance.

Subsequently, while the buffer solution is supplied to the sensor unit 7, the immunoglobulin-containing liquid supply part 54 supplies an immunoglobulin-containing liquid to the liquid storage part 45 of the sensor unit 7. At this time, the immunoglobulin-containing liquid passes through the liquid storage part 45 and is discharged through the liquid discharge pipe 88b. The immunoglobulin 14 contained in the immunoglobulin-containing liquid is adsorbed by the protein forming the base layer 12 on the excitation electrode 22 of the quartz resonator 2 (FIG. 7(a)). The immunoglobulin 14 does not react with the blocker 13 and therefore reacts only with the base layer 12.

Next, the sample solution is supplied to the liquid storage part 45 of the sensor unit 7. A method of supplying the sample solution is as follows. The supply liquid switching part denoted by reference 63 in FIG. 6 includes a valve, which switches the flow channels, and a column, and by the switching of the valve, it is possible to produce a state where one end of the column is connected to the sample solution supply part 55 via the supply channel 57c and the other end of the column is connected to a discharge channel, not shown. In this state, the sample solution is supplied from the sample solution supply part 55 to be stored in the column, and next, by switching the valve, a state is produced where the one end of the column is connected to the supply channel 57a of the buffer solution and the other end of the column is connected to the sensor unit 7. Then, the sample solution in the column is pushed out to the sensor unit 7 by the buffer solution. At this time, the sample solution passes through the liquid storage part 45 and is discharged through the liquid discharge pipe 88b. A substance to be sensed contained in the sample solution is captured by the immunoglobulin 14 formed on the front surface of the adsorption layer 10 of the quartz resonator 2 (FIG. 7(b)).

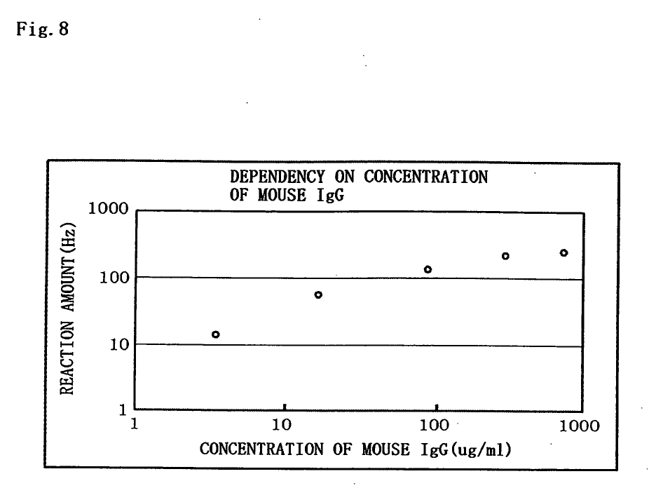

The quartz resonator 2 provided in the sensor unit 7 is oscillated by the oscillator circuit 50 and its oscillation output (frequency signal) is sent to the measuring circuit part 51. The measuring circuit part 51 analog/digital-converts (A/D-converts) the obtained frequency signal and applies the predetermined signal processing to the resultant signal to measure the frequency and also output a measured value of the frequency to the display device 52. As is seen in FIG. 8, which shows an example of frequency data, the frequency is mostly stable when the buffer solution is supplied, but lowers when the sample solution is supplied since the antigen 15 as the substance to be sensed in the sample solution is captured by the immunoglobulin 14. A decremental amount of the frequency corresponds to the concentration of the antigen 15 in the sample solution, and therefore, when, for example, a calibration curve showing a correlation between the concentration of the antigen 15 in the sample solution and the decremental amount of the frequency is prepared, one plot is obtained.

Here, in order to find a variation of the natural frequency of the quartz resonator 2 corresponding to the concentration of the antigen 15, it is necessary to find a difference between an oscillation frequency of the quartz resonator 2 when it is put in the buffer solution and an oscillation frequency of the quartz resonator 2 when it is put in the sample solution, but the method of finding the difference between the oscillation frequencies of the quartz resonator 2 is not limited to the measurement of the oscillation frequencies themselves, but the oscillation frequency difference may be found in such a manner that, for example, a difference frequency between an output frequency of the quartz resonator 2 and a predetermined clock frequency is found, this frequency is evaluated as the frequency of the quartz resonator 2, and a difference between the difference frequencies in the both environments is found.

Subsequently, the acid liquid supply part 58 supplies the sensor unit 7 with an acid liquid, for example, glycine. The acid liquid is supplied to the liquid storage part 45 in the sensor unit 7 via the supply channel 57d, the supply liquid switching part 63, and the liquid supply pipe 88a, and is further discharged from the liquid storage part 45 to the waste liquid storage part 56 via the liquid discharge pipe 88b, the pump 62, and the discharge channel 57e. Then, the acid liquid comes into contact with the immunoglobulin 14 which forms the adsorption layer 10 of the quartz resonator 2 and has captured the substance to be sensed, so that the immunoglobulin 14 is separated from the protein 12 (FIG. 7(c)). As a result, the state on the front surface of the quartz resonator 2 returns again to the state where the film made of the protein 12 and the blocker 13 is formed.

Thereafter, the base layer 12 made of protein is again made to adsorb the immunoglobulin 14 in the above-described manner, whereby the adsorption layer 10 is formed, and then the frequency is measured while the sample solution is supplied to the piezoelectric sensor in the same manner. In this example, the process of supplying only the buffer solution at the time of the switching of the liquids is provided in a series of the processes, so that the previous liquid is pushed out from the inside of the piezoelectric sensor and in this state, the next liquid is supplied into the piezoelectric sensor. A cycle from the supply of the immunoglobulin 14 to the supply of the sample solution, and then to the supply of the acid liquid is automatically performed by, for example, the control unit 100, but the sample solution is changed by a worker.

Further, in the one example, the sample solution supply part 55 is changed every cycle, that is, a container for use is changed among a plurality of containers containing sample solutions different in the concentration of the antigen 15, but another possible structure may be to provide a flow rate regulating part and a flowmeter in each of the supply channels and adjust the concentration of the sample solution supplied into the piezoelectric sensor by adjusting a flow rate ratio of the buffer solution and the sample solution. By thus changing the concentration of the antigen 15 in the sample solution from cycle to cycle and repeating this cycle, it is possible to find the correlation between the concentration of the antigen 15 as the substance to be sensed in the sample solution and the aforesaid frequency difference (difference between the frequency of the quartz resonator 2 corresponding to the buffer solution and the frequency of the quartz resonator 2 corresponding to the sample solution), that is, it is possible to prepare the calibration curve. Such a calibration curve can be used when a user measures the concentration of the antigen 15 in the sample solution.

In the above-described embodiment, by utilizing the property that the immunoglobulin 14 separates from specific protein when coming into contact with the acid liquid, the immunoglobulin 14 is adsorbed by the base layer 12 made of protein which is formed in advance on the excitation electrode, and after the immunoglobulin 14 captures the antigen 15 as the substance to be sensed, the immunoglobulin 14 is brought into contact with the acid liquid to be separated from the base layer 12. Therefore, by repeating a series of these processes, the old adsorption layer 10 which has reacted with the antigen 15 is separated and a new adsorption layer 10 is reproduced.

As a result, it is possible to successively measure samples by using one piezoelectric sensor, which makes it possible not only to save the trouble of changing the piezoelectric sensor every time the measurement is conducted but also to reduce running cost.

The above-described example is an example where the present invention is applied to the preparation of the calibration curve showing the correspondence between the concentration of the antigen 15 and a variation of the frequency of the quartz resonator 2, but another possible example is to find a variation of the frequency of the quartz resonator 2 and detect the concentration of the antigen 15 in the sample solution based on the variation and the calibration curve prepared in advance.

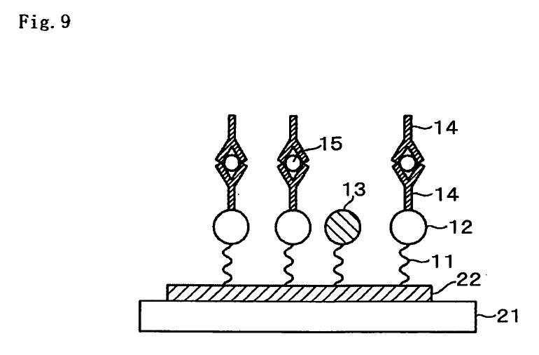

Further, a method to be described next may be adopted as another embodiment. After the base layer 12 adsorbs the immunoglobulin 14, the frequency is measured in the state where the environment in which the quartz resonator 2 is put is replaced by the buffer solution. Thereafter, the sample solution containing the antigen 15 is supplied to the quartz resonator 2 so that the antigen 15 is captured by the immunoglobulin 14 adsorbed by the base layer 12. Next, the liquid containing the immunoglobulin 14 is supplied to the quartz resonator 2 so that the captured antigen 15 adsorbs the immunoglobulin 14 (FIG. 9), and thereafter the frequency is obtained. By finding a difference between the frequency in the buffer solution environment and the obtained frequency, it is possible to prepare the calibration curve. This is because, when the frequency is thus measured after the antigen 15 captured by the immunoglobulin 14 formed on the base layer further adsorbs the immunoglobulin 14, a variation of the frequency corresponding to a captured amount of the antigen 15 becomes large since the immunoglobulin 14 is larger in molecular weight than the antigen 15, which improves measurement accuracy.

Alternatively, the substance to be sensed may be the immunoglobulin 14 instead of the antigen 15. Specifically, if an adsorption amount of the immunoglobulin 14 adsorbed by the base layer 12 is constant, a variation of the oscillation frequency of the quartz resonator 2 depends on the concentration of the antigen 15 in the sample solution, but if conversely the concentration of the antigen 15 in a liquid containing the antigen 15 (corresponding to the sample solution in the above-described example) is constant, a variation of the oscillation frequency of the quartz resonator 2 depends on an adsorption amount of the immunoglobulin 14 adsorbed by the base layer 12. That is, the larger the adsorption amount of the immunoglobulin 14, the larger a captured amount of the antigen 15, and the smaller the adsorption amount of the immunoglobulin 14, the smaller the captured amount of the antigen 15. The adsorption amount of the immunoglobulin 14 corresponds to the concentration of the immunoglobulin 14 in the immunoglobulin-containing liquid, and the specific antigen 15 is captured by the specific immunoglobulin 14. Consequently, it is possible to find the concentration of the specific immunoglobulin 14 in the liquid containing the immunoglobulin 14, by finding a frequency change of the quartz resonator corresponding to the amount of the antigen 15 captured by the immunoglobulin 14.

Therefore, the sample solution in this case is the immunoglobulin-containing liquid. In the case where the immunoglobulin 14 is the substance to be sensed, by using various sample solutions different in concentration of the immunoglobulin 14 and finding a difference between the oscillation frequency of the quartz resonator 2 when it is put in the buffer solution and the oscillation frequency of the quartz resonator 2 when it is put in the liquid containing the antigen 15, it is also possible to prepare a calibration curve showing the correspondence between the difference and the concentration of the immunoglobulin 14. Alternatively, by finding the aforesaid frequency difference using a sample solution whose concentration of the immunoglobulin 14 is not known, it is possible to know the concentration of the specific immunoglobulin 14 capturing the antigen 15 based on the frequency difference and the calibration curve prepared in advance.

The above-described instrument is also usable in the case where the immunoglobulin 14 is thus the substance to be sensed. For example, when the calibration curve is prepared, the concentration of the antigen 15 is set constant instead of changing the concentration of the antigen 15 at each cycle in a series of the above-described processes, and the concentration of the immunoglobulin 14 in the immunoglobulin-containing liquid is changed.

Further, the structure described below may be adopted besides the above-described embodiments. The liquid containing the immunoglobulin 14 is used as the sample solution, and after the base layer 12 adsorbs the immunoglobulin 14, the frequency corresponding to the oscillation frequency of the oscillator circuit is obtained in the state where the environment in which the quartz resonator 2 is put is replaced by the buffer solution. Thereafter, a liquid containing the antigen 15 with constant concentration is supplied to the quartz resonator 2 so that the antigen 15 is captured by the immunoglobulin 14. Next, the sample solution containing the immunoglobulin 14 is supplied so that the immunoglobulin 14 is adsorbed by the captured antigen 15, and thereafter the frequency is obtained. By finding a difference between the frequency obtained in the buffer solution environment and this obtained frequency, it is possible to prepare the calibration curve showing the correlation between the concentration of the immunoglobulin 14 in the sample solution and the frequency difference. When the captured antigen 15 further adsorbs the immunoglobulin 14, a variation of the frequency of the quartz resonator 2 corresponding to the adsorption amount of the antigen 15 becomes large, so that an accurate calibration curve can be prepared. In this case, the liquid which is added in order to cause the antigen 15 captured by the immunoglobulin to further adsorb the immunoglobulin may be a liquid whose concentration of the immunoglobulin is known in advance, instead of the sample solution.

As described above, in the present invention, it is possible to find the concentration of the antigen 15 and the concentration of the immunoglobulin 14, and therefore, the present invention is an art effective for a blood test or the like, for instance (the sample solution is blood).

FIG. 8 shows one example of the calibration curve showing the correlation, which is obtained by using the above-described sensing instrument, between the concentration of the immunoglobulin 14 in the immunoglobulin-containing liquid and the frequency difference (difference between the oscillation frequency of the quartz resonator 2 when it is put in the buffer solution and the oscillation frequency of the quartz resonator 2 when it is put in the liquid containing the antigen 15), and mouse IgG is used as the immunoglobulin 14.

1. A sensing method in which a piezoelectric resonator having an excitation electrode formed on a piezoelectric piece is oscillated by an oscillator circuit while being in contact with a liquid and an antigen in a sample solution is sensed based on a change in natural frequency of the piezoelectric resonator,
the piezoelectric resonator being a piezoelectric resonator in which a base layer made of protein adsorbing an immunoglobulin and desorbing the immunoglobulin under an atmosphere of an acid liquid is formed on the excitation electrode, the method comprising:
a step of supplying a liquid containing the immunoglobulin to the piezoelectric resonator to make the base layer adsorb the immunoglobulin;
a first measuring step of oscillating the piezoelectric resonator which has adsorbed the immunoglobulin and measuring a frequency corresponding to an oscillation frequency of the oscillator circuit;
a step, which follows said first measuring step, of supplying the piezoelectric resonator with the sample solution containing the antigen which is a substance to be sensed captured by the immunoglobulin;
a second measuring step of oscillating the piezoelectric resonator which has adsorbed the antigen and measuring a frequency corresponding to an oscillation frequency of the oscillator circuit;
a step of finding a difference between the frequencies obtained in said first measuring step and said second measuring step respectively; and
a step, which follows said step of finding the difference, of supplying the acid liquid to the piezoelectric resonator to make the base layer desorb the immunoglobulin.

2. A sensing method in which a piezoelectric resonator having an excitation electrode formed on a piezoelectric piece is oscillated by an oscillator circuit while being in contact with a liquid and an immunoglobulin in a sample solution is sensed based on a change in natural frequency of the piezoelectric resonator,
the piezoelectric resonator being a piezoelectric resonator in which a base layer made of protein adsorbing the immunoglobulin and desorbing the immunoglobulin under an atmosphere of an acid liquid is formed on the excitation electrode, the method comprising:
a step of supplying the piezoelectric resonator with the sample solution containing the immunoglobulin which is a substance to be sensed to make the base layer adsorb the immunoglobulin;
a first measuring step of oscillating the piezoelectric resonator which has adsorbed the immunoglobulin and measuring a frequency corresponding to an oscillation frequency of the oscillator circuit;
a step, which follows said first measuring step, of supplying the piezoelectric resonator with a liquid containing an antigen captured by the immunoglobulin;
a second measuring step of oscillating the piezoelectric resonator which has adsorbed the antigen and measuring a frequency corresponding to an oscillation frequency of the oscillator circuit;
a step of finding a difference between the frequencies obtained in said first measuring step and said second measuring step respectively; and
a step, which follows said step of finding the difference, of supplying the acid liquid to the piezoelectric resonator to make the base layer desorb the immunoglobulin.

3. The sensing method according to claim 1, further comprising
a step of supplying a liquid containing the immunoglobulin to the piezoelectric resonator which has adsorbed the antigen to make the antigen adsorb the immunoglobulin, so as to sandwich the antigen between the immunoglobulin and the immunoglobulin which has already adsorbed the antigen, wherein
said second measuring step is a step of measuring the frequency while the antigen is sandwiched by the immunoglobulins.

4. The sensing method according to claim 1, wherein a self-assembled monolayer is formed between the excitation electrode and the base layer.

5. The sensing method according to claim 1, wherein
a blocking substance for preventing the adsorption of the antigen is adsorbed by a portion, on an excitation electrode, which has not adsorbed the immunoglobulin.

6. The sensing method according to claim 2, further comprising
a step of supplying a liquid containing the immunoglobulin to the piezoelectric resonator which has adsorbed the antigen to make the antigen adsorb the immunoglobulin, so as to sandwich the antigen between the immunoglobulin and the immunoglobulin which has already adsorbed the antigen, wherein
said second measuring step is a step of measuring the frequency while the antigen is sandwiched by the immunoglobulins.

7. The sensing method according to claim 2, wherein a self-assembled monolayer is formed between the excitation electrode and the base layer.

8. The sensing method according to claim 2, wherein
a blocking substance for preventing the adsorption of the antigen is adsorbed by a portion, on an excitation electrode, which has not adsorbed the immunoglobulin.
